(19) United States
(12) Patent Application Publication
(10) Pub. No.: US 2013/0017616 A1
(43) Pub. Date: Jan. 17, 2013

(54) Methods For Treating Inflammation

(71) Applicant: Billy G. Hudson, Brentwood, TN (US)

(72) Inventor: Billy G. Hudson, Brentwood, TN (US)

(73) Assignee: Vanderbilt University - Center for Technology Transfer and Commercialization, Nashville, TN (US)

(21) Appl. No.: 13/548,662

(22) Filed: Jul. 13, 2012

Related U.S. Application Data
(60) Provisional application No. 61/508,466, filed on Jul. 15, 2011.

(51) Int. Cl. A61K 31/44; G01N 21/75; A61P 25/16; A61P 1/00; A61P 29/00; A61P 11/06

(52) U.S. Cl. 436/96; 514/351

(57) ABSTRACT

The present invention provides methods for treating or limiting development of inflammatory disorders.

### CROSS REFERENCE

This application claims priority to U.S. Provisional Application Ser. No. 61/508466 filed Jul. 15, 2011, incorporated herein by reference in its entirety.

### STATEMENT OF GOVERNMENT RIGHTS

This invention was made with government support under grant number DK65138 awarded by National Institutes of Health. The government has certain rights in the invention

### BACKGROUND

Hypohalous acids, such as hypochlorous acid (HOCl) and hypobromous acid (HOBr) are mediators of the immune response. HOCl is generated in cells including activated neutrophils by myeloperoxidase (MPO)-mediated peroxidation, and in eosinophils by eosinophil peroxidase (EPO)-mediated peroxidation, of chloride ions. (See, for example, Davies et al., Antioxidants & Redox Signaling, Volume 10, Number 7, 2008, p. 1199). HOBr is generated in cells including activated eosinophils by EPO-mediated peroxidation of bromide ions.

Hypohalous acids such as HOCl and HOBr can react avidly with nucleophiles, especially those containing sulfur or nitrogen atoms, such as thiols, thioethers, amines, and amides, and thus reacts with a wide variety of biomolecules including DNA, RNA, fatty acid groups, cholesterol, and proteins. Such reactions lead to the formation of toxic reactive oxygen and carbonyl species that have been implicated in a wide variety of inflammatory disorders. Thus, methods that serve to scavenge hypohalous acids such as HOCl and HOBr before such reactive species can be formed can be used to treat a wide variety of inflammatory disorders, or to limit development of the inflammatory disorder in subjects at risk of developing the inflammatory disorder.

### SUMMARY OF THE INVENTION

In a first aspect, the present invention provides methods for treating an inflammatory disorder, comprising administering to a subject suffering from an inflammatory disorder thereof an amount of pyridoxamine, or a pharmaceutically acceptable salt thereof, to treat the inflammatory disorder. In another aspect, the present invention provides methods for limiting development of an inflammatory disorder, comprising administering to a subject at risk of developing an inflammatory disorder an amount of pyridoxamine, or a pharmaceutically acceptable salt thereof, to limit development of the inflammatory disorder.

In a third aspect, the present invention provides methods for treating or limiting development of an inflammatory disorder, comprising:

(a) identifying a subject with an inflammatory disorder that produces excess hypohalous acid compared to control; and

(b) treating the subject that produces excess hypohalous acid with an amount of pyridoxamine, or a pharmaceutically acceptable salt thereof, to treat the treat or limit development of the inflammatory disorder. In various preferred embodiments, the hypohalous acid is selected from the group consisting of hypochlorous acid (HOCl) and hypobromous acid (HOBr).

In a further aspect, the present invention provides methods for monitoring pyridoxamine therapy in a subject, comprising

(a) analyzing a urine sample obtained from a subject with an inflammatory disorder being treated with pyridoxamine, or a pharmaceutically acceptable salt thereof, for the presence of pyridoxamine adducts with hypohalous acids; and

(b) comparing an amount of pyridoxamine-hypohalous adducts in the sample against a standard; and

(c) determining efficacy of the pyridoxamine therapy to treat the inflammatory disorder based on the comparison.

In various embodiments of each aspect of the invention, the subject has, or is at risk of developing, an inflammatory disorder selected from the group consisting of cystic fibrosis, asthma, rheumatoid arthritis, multiple sclerosis, Parkinson's disease, inflammatory bowel disease, irritable bowel syndrome, ulcerative colitis, vasculitis, and Crohn's disease.

### DESCRIPTION OF THE FIGURES

FIG. 1. HOCl and HOBr inhibit RNase activity while PM has no effect on activity. RNase (15 μg/mL) was incubated alone or with different concentrations of HOCl, HOBr or PM at 37° C. for 2 h. Enzymatic activity was determined as described under Experimental Procedures.

FIG. 2. Relative reactivity of PM with either HOCl or HOBr compared to different amino acid side chains. RNase (15 μg/mL) was incubated alone, with 60 μMor either HOCl or HOBr, or with 60 μM of either HOCl or HOBr and 60 μM of different additives at 37° C. for 2 h. RNase activity was determined as described under Experimental Procedures. Numbers above the bars indicate IC₅₀ values in μM.

FIG. 3A. Modification of functionally important chlorinated Tyr species (Y-92 and Y-97) in RNase by HOCl and protection by PM. *P<0.05, control vs. HOCl (n=3); **P<0.05, HOCl vs. HOCl+PM (n=3).

FIG. 3B. Modification of functionally important chlorinated Tyr species (Y-92 and Y-97) in RNase by HOBr and protection by PM. *P<0.05, control vs. HOBr; **P<0.05, HOBr vs. HOBr+PM.

FIG. 4A. Identities of PM reaction products with HOCl. 0.5 mM HOCl and 0.5 mM PM were incubated in 100 mM sodium phosphate buffer in the dark at 37° C. for the indicated times. The aliquot (10 μL) was loaded onto Phenomenex Synergy 4u Hydro-RP 80A (150×4.6 mm) column and eluted at a flow rate of 1 ml/min with the following gradient: 0-5 min, 100% buffer A; 5-15 min, linear gradient to 25% buffer B; 15-15.1 min, change to 100% buffer B; 15.1-20 min, 100% buffer B; 20-20.1 min, change to 100% buffer A; 20.1-25 min, 100% buffer A. Buffer A was 0.5% formic acid in water, buffer B was 0.5% formic acid in ACN. The UV detector was set at 295 nm. The selected reaction products were identified by LC-MS/MS using ThermoScientific TSQ mass spectrometer as described under Experimental Procedures.

FIG. 4B. Identities of PM reaction products with HOBr. 0.5 mM HOBr and 0.5 mM PM were incubated in 100 mM sodium phosphate buffer in the dark at 37° C. for the indicated times. The aliquot (10 μL) was analyzed as described in FIG. 4A. The selected reaction products were identified by LC-MS/MS using ThermoScientific TSQ mass spectrometer as described under Experimental Procedures.

FIG. 5. Chlorination of ECM proteins by HOCl and protection by PM. EHS collagen IV was coated onto 96-well plates; ECM derived from PHFR-9 cells was deposited onto 6-well plates and cell were removed as described under Experimental Procedures. After washing, ECM proteins were incubated with the indicated concentrations of either HOCl or HOCl with or without the equimolar concentrations of PM for 1 h at 37° C. Protein chloramine content was determined as described under Experimental Procedures. *P<0.05, control vs. HOCl; **P<0.05, HOCl vs. HOCl+PM (n=4).

FIG. 6A. EHS Collagen IV was coated onto 96-well plate at 20 μg/mL, washed and incubated with the indicated concentrations of HOCl for 1 h at 37° C. Binding of α1β1 integrin was determined after 2 h at 30° C. using solid phase binding assay as described under Experimental Procedures. *P<0.05, control vs. HOCl (n=4).

FIG. 6B. EHS Collagen IV was coated onto 96-well plate overnight at 4° C. Collagen IV was incubated with 0.1 mM HOCl or HOBr either with or without 0.1 mM PM for 1 h at 37° C. Collagen IV was then incubated with 1 μg/mL of integrin α1β1 for 2 h at 30° C. Integrin binding was detected in solid phase binding assay using integrin α1β1 antibody. The bars represent background-subtracted Mn²⁺⁻ dependent binding±SD (n=4). *P<0.05, control vs. HOCl or HOBr; **P<0.05, HOCl or HOBr vs. HOCl+PM or HOBr+PM.

### DETAILED DESCRIPTION OF THE INVENTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise. “And” as used herein is interchangeably used with “or” unless expressly stated otherwise.

All embodiments of any aspect of the invention can be used in combination, unless the context clearly dictates otherwise.

In a first aspect, the present invention provides methods for treating an inflammatory disorder, comprising administering to a subject suffering from an inflammatory disorder thereof an amount of pyridoxamine, or a pharmaceutically acceptable salt thereof, to treat the inflammatory disorder.

In a second aspect, the present invention provides methods for limiting development of an inflammatory disorder, comprising administering to a subject at risk of developing an inflammatory disorder an amount of pyridoxamine, or a pharmaceutically acceptable salt thereof, to limit development of the inflammatory disorder.

The inventors have shown herein for the first time that pyridoxamine can be used to scavenge hypohalous acids, such as hypochlorous acid (HOCl) and hypobromous acid (HOBr), mediators of the immune response. HOCl is generated in cells including activated neutrophils by myeloperoxidase (MPO)-mediated peroxidation, and in eosinophils by eosinophil peroxidase (EPO)-mediated peroxidation, of chloride ions. HOBr is generated in cells including activated eosinophils by EPO-mediated peroxidation of bromide ions. Hypohalous acids such as HOCl and HOBr react avidly with nucleophiles, especially those containing sulfur or nitrogen atoms, such as thiols, thioethers, amines, and amides, and thus reacts with a wide variety of biomolecules including DNA, RNA, fatty acid groups, cholesterol, and proteins. Such reactions lead to the formation of toxic reactive oxygen and carbonyl species that have been implicated in a wide variety of inflammatory disorders. Thus, methods that serve to scavenge HOCl before such reactive species can be formed can be used to treat a wide variety of inflammatory disorders, or to limit development of the inflammatory disorder in subjects at risk of developing the inflammatory disorder.

As used herein, “treat” or “treating” means accomplishing one or more of the following: (a) reducing the severity of the inflammatory disorder; (b) limiting or preventing development of symptoms characteristic of the inflammatory disorder(s) being treated; (c) inhibiting worsening of symptoms characteristic of the inflammatory disorder(s) being treated; (d) limiting or preventing recurrence of the inflammatory disorder(s) in patients that have previously had the disorder(s); and (e) limiting or preventing recurrence of symptoms in patients that were previously symptomatic for the inflammatory disorder(s).

As used herein, the term “limit” or “limiting” means to limit development of the disorder in individuals at risk of developing the disorder, including but not limited to subjects that are genetically predisposed to an inflammatory disorder, have family members that suffer from the inflammatory disorder, or are otherwise exposed to risk factors for developing the inflammatory disorder.

The subject may be any suitable subject, preferably a human subject. In a preferred embodiment, the subject is a human subject and does not suffer from diabetic nephropathy.

The subject may be suffering from, or at risk of developing any inflammatory disorder that is impacted by production of hypohalous acids (such as HOCL and/or HOBr), including but not limited to atherosclerosis, vasculitis, cardiovascular disease, retinopathy, cystic fibrosis, asthma, rheumatoid arthritis, glomerulonephritis, multiple sclerosis, Parkinson's disease, inflammatory bowel disease, irritable bowel syndrome, ulcerative colitis, and Crohn's disease. In a preferred embodiment, the inflammatory disorder is selected from the group consisting of cystic fibrosis, asthma, rheumatoid arthritis, multiple sclerosis, Parkinson's disease, inflammatory bowel disease, irritable bowel syndrome, ulcerative colitis, and Crohn's disease.

Atherosclerosis is a chronic inflammatory response in the walls of arteries, caused largely by the accumulation of macrophages and promoted by low-density lipoproteins without adequate removal of fats and cholesterol from the macrophages by high density lipoproteins. Symptoms of atherosclerosis include, but are not limited to, presence of atherosclerotic lesions in the vessels, stenosis, ischemia, and aneurysm.

Cystic fibrosis is a genetic disorder of the cystic fibrosis transmembrane conductance regulator (CFTR) gene, which is characterized by abnormal ion transport across epithelial membranes, leading to thick, viscous secretions. Symptoms of cystic fibrosis include, but are not limited to fibrosis and cyst formation in the pancreas, breathing difficulties, sinus infections, and thick, viscous secretions in the lungs.

Asthma is a common chronic inflammatory airway disease characterized by variable and recurring symptoms, reversible airflow obstruction, and bronchospasm. Symptoms include, but are not limited to wheezing, coughing, chest tightness, and shortness of breath. Asthma is thought to be caused by a combination of genetic and environmental factors.

Rheumatoid arthritis is a chronic systemic inflammatory disorder that may affect many tissues and organs, but principally attacks synovial joints. The process involves an inflammatory response of the synovial capsule around the joints secondary to hyperplasia of synovial cells, excess synovial fluid, and the development of fibrous tissue in the synovia. The pathology of the disease process often leads to the destruction of articular cartilage and ankylosis of the joints. Rheumatoid arthritis can also produce diffuse inflammation in the lungs, pericardium, lung pleura, sclera, and nodular lesions, most common in subcutaneous tissue. Although the cause of rheumatoid arthritis is unknown, autoimmunity plays a pivotal role in both its chronicity and progression. About 1% of the world's population is afflicted by rheumatoid arthritis, women three times more often than men. Onset is most frequent between the ages of 40 and 50, but people of any age can be affected. In addition, individuals with the HLA-DR1 or HLA-DR4 serotypes have an increased risk for developing the disorder.

Glomerulonephritis is a renal disease characterized by inflammation of the glomeruli. Symptoms include, but are not limited to inflammation of the glomeruli, hematuria and/or proteinuria.

Multiple sclerosis (MS) is an inflammatory disease involving demyelination of myelin sheaths surrounding brain and spinal cord axons. MS symptoms include, but are not limited to scarring of white matter in the brain and/or spinal cord and a wide variety of neurological symptoms, including but not limited to changes in sensation such as loss of sensitivity or tingling, pricking or numbness (hypoesthesia and parasthesia), muscle weakness, clonus, muscle spasms or difficulty in moving; difficulties with coordination and balance (ataxia); problems in speech (dysarthria) or swallowing (dysphagia), visual problems (nystagmus, optic neuritis, etc.), fatigue, acute/chronic pain, and bladder and bowel difficulties. Cognitive impairment of varying degrees and depression are also common. Symptoms of MS usually appear in episodic acute periods of worsening in a gradually progressive deterioration of neurologic function, or in a combination of both. Multiple sclerosis relapses are often unpredictable, occurring without warning and without obvious inciting factors with a rate rarely above one and a half per year. Some attacks, however, are preceded by common triggers. Relapses occur more frequently during spring and summer. Viral infections such as the common cold, influenza, or gastroenteritis increase the risk of relapse. Stress may also trigger an attack. Pregnancy affects the susceptibility to relapse, with a lower relapse rate at each trimester of gestation. During the first few months after delivery, however, the risk of relapse is increased.

The motor symptoms of Parkinson's disease (PD) result from the death of dopamine-generating cells in the substantia nigra, a region of the midbrain; the cause of this cell death is unknown. Early in the course of the disease, the most obvious symptoms are movement-related; these include shaking, rigidity, slowness of movement and difficulty with walking and gait. Later, cognitive and behavioral problems may arise, with dementia commonly occurring in the advanced stages of the disease. Other symptoms include sensory, sleep and emotional problems. PD is more common in the elderly, with most cases occurring after the age of 50.

Inflammatory bowel disease (IBD) is a group of inflammatory conditions of the colon and small intestine. The major types of IBD are Crohn's disease and ulcerative colitis (see below). Other types of IBD include collagenous colitis, lymphocytic colitis, ischemic colitis, diversion colitis, Behcet's disease, and indeterminate colitis.

Irritable bowel syndrome is a symptom-based diagnosis characterized by chronic abdominal pain, discomfort, bloating, and alteration of bowel habits. Diarrhea or constipation may predominate, or they may alternate. Onset of IBS is more likely to occur after an infection, a stressful life event, or onset of maturity.

Ulcerative colitis is an IBD of the colon that includes characteristic ulcers, or open sores. The main symptom of active disease is usually constant diarrhea mixed with blood, of gradual onset. Ulcerative colitis is an intermittent disease, with periods of exacerbated symptoms, and periods that are relatively symptom-free. Although the symptoms of ulcerative colitis can sometimes diminish without treatment, the disease usually requires treatment to go into remission. Although ulcerative colitis has no known cause, there is a presumed genetic component to susceptibility. The disease may be triggered in a susceptible person by environmental factors. Although dietary modification may reduce the discomfort of a person with the disease, ulcerative colitis is not thought to be caused by dietary factors.

Crohn's disease is an IBD that may affect any part of the gastrointestinal tract, causing a wide variety of symptoms. It primarily causes abdominal pain, diarrhea (which may be bloody if inflammation is at its worst), vomiting (can be continuous), or weight loss, but may also cause complications outside the gastrointestinal tract such as skin rashes, arthritis, eye inflammation, tiredness, and lack of concentration. There is a genetic association with Crohn's disease, primarily with variations of the NOD2 gene and its protein. Siblings of affected individuals are at higher risk. Males and females are equally affected. Smokers are two times more likely to develop Crohn's disease than nonsmokers.

Vasculitis refers to a heterogeneous group of disorders that are characterized by inflammatory destruction (such as neutrophil activation) of blood vessels. According to the size of the vessel affected, vasculitis can be classified into:
Large vessel: Behcet's syndrome, Polymyalgia rheumatica, Takayasu's arteritis, Temporal arteritis;
Medium vessel: Buerger's disease, cutaneous vasculitis. Kawasaki disease, Polyarteritis nodosa; and
Small vessel: Churg-Strauss syndrome, cutaneous vasculitis, Henoch-Schonlein purpura, Microscopic polyangiitis, and Wegener's granulomatosis.

Vasculitis symptoms include, but are not limited to the following:
General symptoms: fever, weight loss;
Skin: Palpable purpura, livedo reticularis;
Muscles and joints: Myalgia or myositis, arthralgia or arthritis;
Nervous system: Mononeuritis multiplex, headache, stroke, tinnitus, reduced visual acuity, acute visual loss;
Heart and arteries: Myocardial infarction, hypertension, gangrene;
Respiratory tract: Nose bleeds, bloody cough, lung infiltrates;
GI tract: Abdominal pain, bloody stool, perforations; and
Kidneys: Glomerulonephritis.

Any suitable amount of pyridoxamine may be administered as is deemed suitable by an attending physician. Exemplary dosage unit forms of the pyridoxamine component of the compositions of the present invention comprise between 25 mg and 1000 mg of pyridoxamine, or a pharmaceutically acceptable salt thereof. Such dosage unit forms can comprise, for example, 25, 50, 75, 100, 125, 150, 175, 200, 250, 300, 350, 400, 450, 500, 550, 600, 650, 700, 750, 800, 850, 900, 950, or 1000 mg of pyridoxamine, or a pharmaceutically acceptable salt thereof, or any range of such dosage unit forms. In a preferred embodiment, the dosage unit forms of the pharmaceutical compositions comprise between 50 mg and 500 mg, 50 mg and 400 mg, or 50 mg and 300 mg of pyridoxamine, or a pharmaceutically acceptable salt thereof. Such dosage unit forms can comprise, for example, 50, 75, 100, 125, 150, 175, 200, 250, 300, 350, 400, 450, or 500 mg of pyridoxamine, or a pharmaceutically acceptable salt thereof. The dosage unit form can be selected to accommodate the desired frequency of administration used to achieve a specified daily dosage of pyridoxamine, or a pharmaceutically acceptable salt thereof to a patient in need thereof. Preferably the unit dosage form is prepared for once daily or twice daily administration to achieve a daily dosage of between 50 and 2000 mg, more preferably between 100 and 1000 milligrams, and even more preferably between 100 and 500 milligrams.

In any of the embodiments herein, the methods may further comprise administering a further anti-inflammatory to provide additional benefit to the subject. Any suitable further anti-inflammatory for the particular subject being treated can be used as deemed appropriate by an attending physician, including but not limited to ibuprofen, celecoxib, aspirin, indomethacin, diclofenac, corticosteroids (ex: prednisone, cortisone, scalacort, hytone cotacort, etc.), Naprosyn, Aflaxen, Indocin, Anaprox, naproxen sodium, etc.

Pharmaceutically acceptable salts in accordance with the present invention are the salts with physiologically acceptable bases and/or acids well known to those skilled in the art of pharmaceutical technique. Suitable salts with physiologically acceptable bases include, for example, alkali metal and alkaline earth metal salts, such as sodium, potassium, calcium and magnesium salts, and ammonium salts and salts with suitable organic bases, such as methylamine, dimethylamine, trimethylamine, piperidine, morpholine and triethanolamine. Suitable salts with physiologically acceptable acids include, for example, salts with inorganic acids such as hydrohalides (especially hydrochlorides or hydrobromides), sulphates and phosphates, and salts with organic acids.

In all aspects of the compositions of the present invention, the compounds are combined with one or more pharmaceutically acceptable carriers appropriate for the indicated route of administration. The compounds may be admixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

In a preferred embodiment, the compositions of the invention are prepared for oral administration. As such, the composition can be in the form of, for example, a tablet, a hard or soft capsule, a lozenge, a cachet, a dispensable powder, granules, a suspension, an elixir, a liquid, or any other form reasonably adapted for oral administration. The compositions can further comprise, for example, buffering agents. Tablets, pills and the like additionally can be prepared with enteric coatings. Unit dosage tablets or capsules are preferred.

Compositions suitable for buccal administration include, for example, lozenges comprising pyridoxamine and sulodexide, or pharmaceutically acceptable salts thereof and a flavored base, such as sucrose, acacia tragacanth, gelatin, and/or glycerin.

Liquid dosage forms for oral administration can comprise pharmaceutically acceptable emulsions, solutions, suspensions, syrups, and elixirs containing inert diluents commonly used in the art, such as water. Such compositions can also comprise, for example, wetting agents, emulsifying and suspending agents, and sweetening, flavoring, and perfuming agents.

In another aspect the present invention provides methods for treating or limiting an inflammatory disorder, comprising administering to a subject that has been identified as producing excess HOCl an amount of pyridoxamine, or a pharmaceutically acceptable salt thereof, to treat the treat or limit the inflammatory disorder.

In a further aspect the present invention provides methods for treating or limiting development of an inflammatory disorder, comprising:

(a) identifying a subject that produces excess hypohalous acid; and

(b) treating the subject that produces excess hypohalous acid with an amount of pyridoxamine, or a pharmaceutically acceptable salt thereof, to treat the treat or limit development of an inflammatory disorder.

Hypohalous acids are any oxyacid (acid containing oxygen) of a halogen of the general formula “HOX”, where X is the halogen. Thus, X can be selected from the group consisting of fluorine (HOF), chlorine (HOCl), bromine (HOBr), iodine (HOI), and astatine (HOAt). In a preferred embodiment, the hypohalous acid is selected from the group consisting of HOCl and HOBr.

As discussed herein, the inventors have shown herein for the first time that pyridoxamine can be used to scavenge hypohalous acids, such as HOCl and HOBr. Since HOCl and HOBr are known as important mediators of the immune response, subjects that are over-producing HOCl and/or HOBr will benefit from pyridoxamine therapy by a treating or limiting development of one or more inflammatory conditions.

The amount of hypohalous acid produced by the subject can be determined from any suitable body sample, including but not limited to urine, whole blood, plasma, serum, saliva, feces, tears, etc. Suitable methods for determining hypohalous acid levels in a body sample are known to those of skill in the art. In a preferred embodiment, the body sample is a urine sample.

As used herein, “excess hypohalous acid” is any amount of hypohalous acid above a normal range. Any suitable control can be used for comparison to the subject's hypohalous acid level, including but not limited to known standard from individual/population and standards generated from previous studies (e.g., positive or negative standards).

In another aspect, the present invention provides methods for monitoring pyridoxamine therapy in a subject, comprising

(a) analyzing a urine sample obtained from a subject being treated with pyridoxamine, or a pharmaceutically acceptable salt thereof, for the presence of pyridoxamine adducts with hypohalous acids; and

(b) comparing an amount of pyridoxamine-hypohalous acid adducts against a standard;

wherein the comparison provides a measure of efficacy of pyridoxamine therapy in the subject.

The inventors have shown herein for the first time that amounts of pyridoxamine-hypohalous acid adducts can be detected in urine, and profiles of such “metabolomic” products generated to provide a measure of the efficacy of pyridoxamine therapy and/or to guide an attending physician in recommending an appropriate dosage of pyridoxamine for a patient. In a preferred embodiment, the hypohalous acid is selected from the group consisting of HOCl and HOBr.

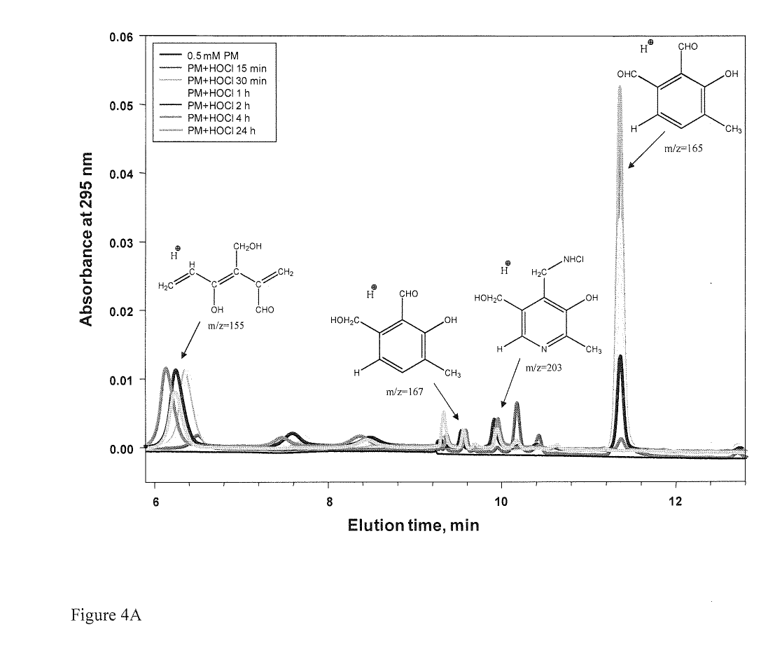

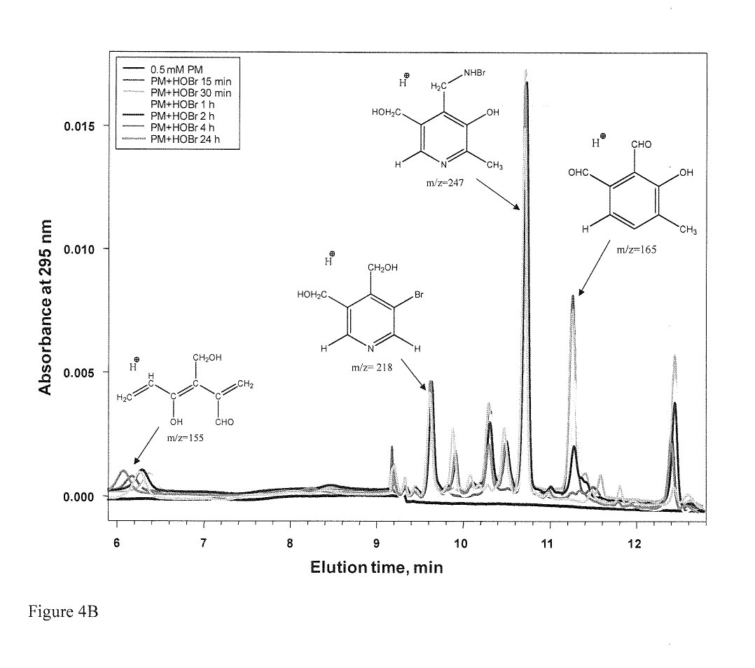

Exemplary pyridoxamine-hypohalous adducts are shown in FIGS. 4A-4B, as shown further below. As will be understood by those of skill in the art, when detecting comprises the use of mass spectrometry, these compounds will be detected as their mass ions (e.g., M+H⁺).

### PYR-HOCl Adducts:

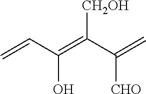

(E)-4-hydroxy-3-(hydroxymethyl)-2-methylenehexa-3,5-dienal

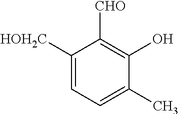

2-hydroxy-6-(hydroxymethyl)-3-methylbenzaldehyde

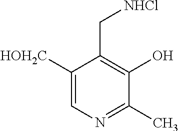

4-((chloroamino)methyl)-5-(hydroxymethyl)-2-methylpyridin-3-ol

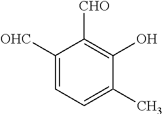

3-hydroxy-4-methylphthalaldehyde

### PYR-HOBr Adducts:

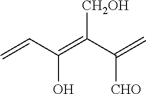

(E)-4-hydroxy-3-(hydroxymethyl)-2-methylenehexa-3,5-dienal

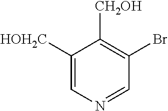 [SMILES: OCc1cncc(Br)c1CO]

(5-bromopyridine-3,4-diyl)dimethanol

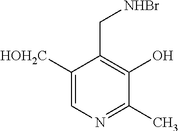

4-((bromoamino)methyl)-5-(hydroxymethyl)-2-methylpyridin-3-ol

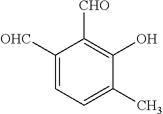

3-hydroxy-4-methylphthalaldehyde

Thus, the methods may comprise detecting one or more of the adducts disclosed herein and comparing to control.

In one embodiment, the analyzing comprises NMR and/or RP-HPLC and/or LC-MS (liquid chromatography mass spectrometry) analysis of urine sample for one or more of the pyridoxamine adducts. Any suitable control can be used, including but not limited to comparisons with urine samples from (a) untreated individual/population; (b) the subject at earlier time points of the treatment (ex: before treatment; after several doses, etc.), and (c) standards generated from previous studies (e.g., positive or negative standards).

In this aspect, the subject may be any subject being treated with pyridoxamine, including those with inflammatory disease, as well as those being treated for other disorders, including but not limited to diabetic nephropathy. The methods can be used to monitor the benefits of pyridoxamine therapy, for example, to determine whether an increase or decrease in pyridoxamine dosage should be recommended for the patient, or if a recommendation to halt pyridoxamine therapy should be considered.

### EXAMPLES

### Mechanism of Action of Pyridoxamine: Protection of Protein Function by Scavenging of Toxic Hypohalous Acids

With an estimated 485 million people affected by 2030, diabetes has become a worldwide pandemic. With 30-40% of diabetics being affected by nephropathy, end stage renal disease (ESRD) is of great concern given the requirement for dialysis or kidney transplantation to sustain the patient's life. There is a pressing need to develop novel therapeutics for preventing or delaying the progression to diabetic ESRD.

Among the established pathogenic mechanisms of diabetic nephropathy (DN) is the activation of oxidative pathways which promote micro vascular disease. One of these pathways is increase of production of hypohalous acids by the family of peroxidase enzymes in diabetes. Vascular-bound myeloperoxidase (MPO) is activated in diabetes causing overproduction of hypochlorous acid and increased chlorination of renal proteins has been found in patients with kidney disease [1-3]. Resulting protein damage is thought to be a contributing factor to microvascular dysfunction and fibrosis [2]. In particular, MPO-induced chlorination has been shown to uncouple and inhibit endothelial nitric oxide synthase (eNOS) and exacerbate oxidative stress [4-5]. MPO can also produce hypobromous acid under physiological bromine concentrations, thus causing protein bromination [6]. Mammalian vascular peroxidase l/peroxidasin, an extracellular matrix (ECM)-bound peroxidase, has also been shown to contribute to microvascular disease and renal fibrosis via pathogenic overproduction of hypochlorous acid [7-10].

Pyridoxamine (Pyridorin®), an investigational drug which is approved by the FDA for Phase 3 clinical trials in diabetic nephropathy, possesses activity against several of pathogenic oxidative pathways [11-13]. However, it is unknown whether pyridoxamine (PM) can protect against protein damage induced by hypohalous acids. In the present study, we demonstrated the capacity of PM to inhibit functional protein damage caused by hypohalous acids using ribonuclease (RNase), purified collagen IV and native ECM as model systems.

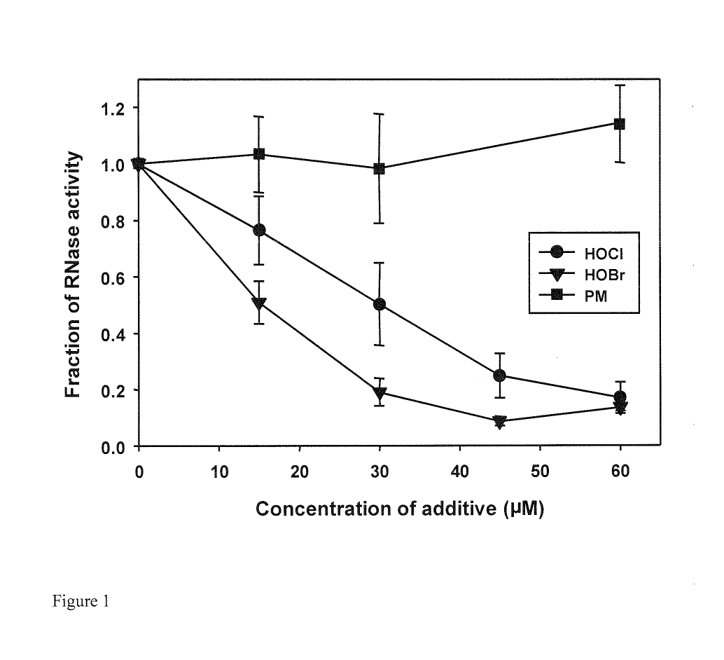

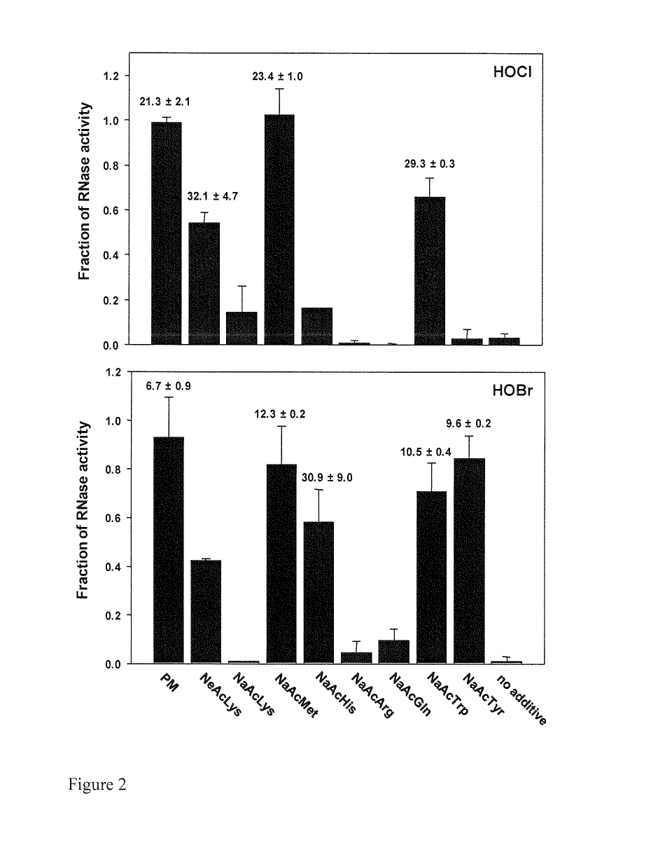

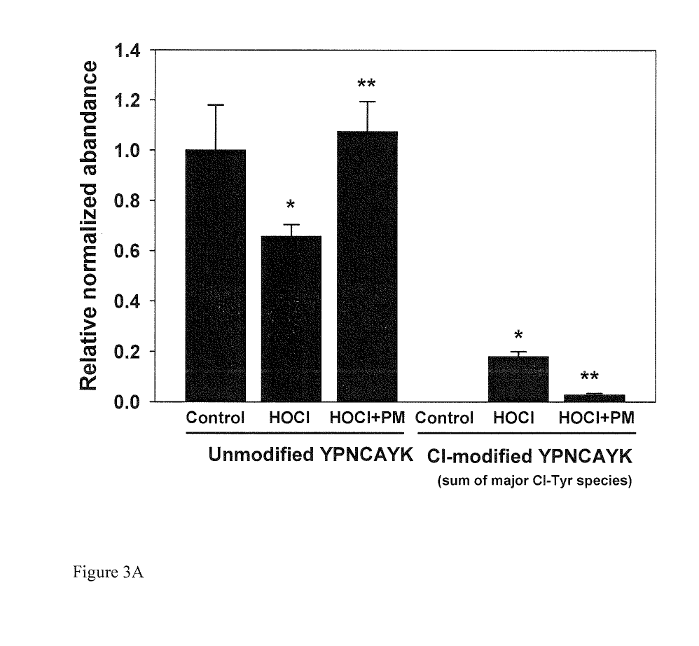

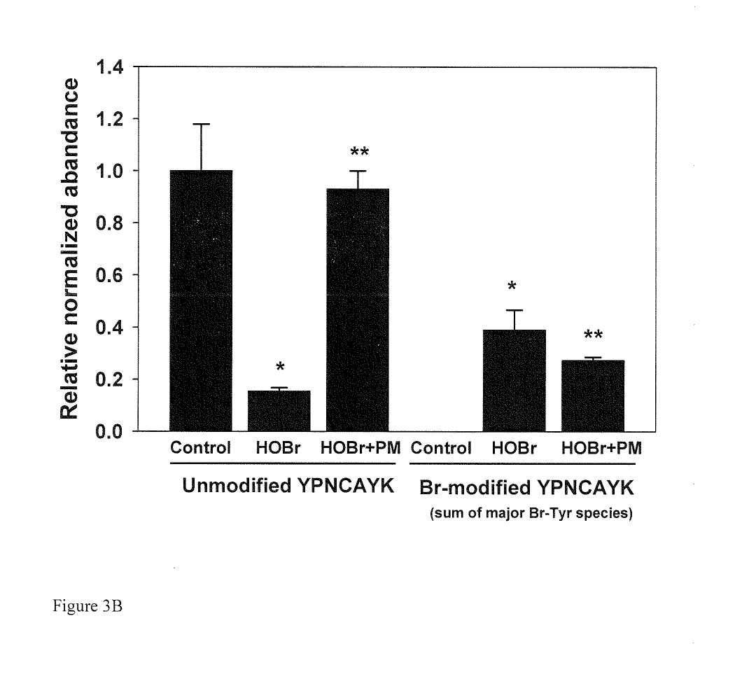

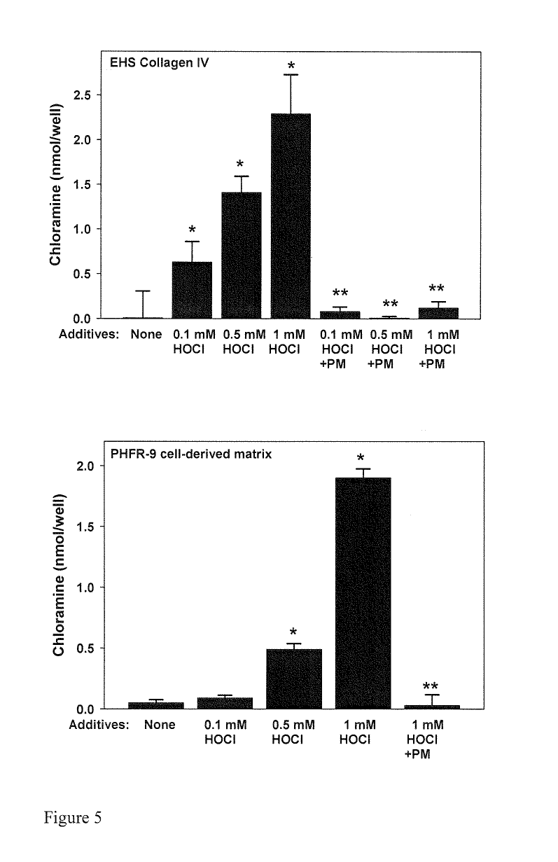

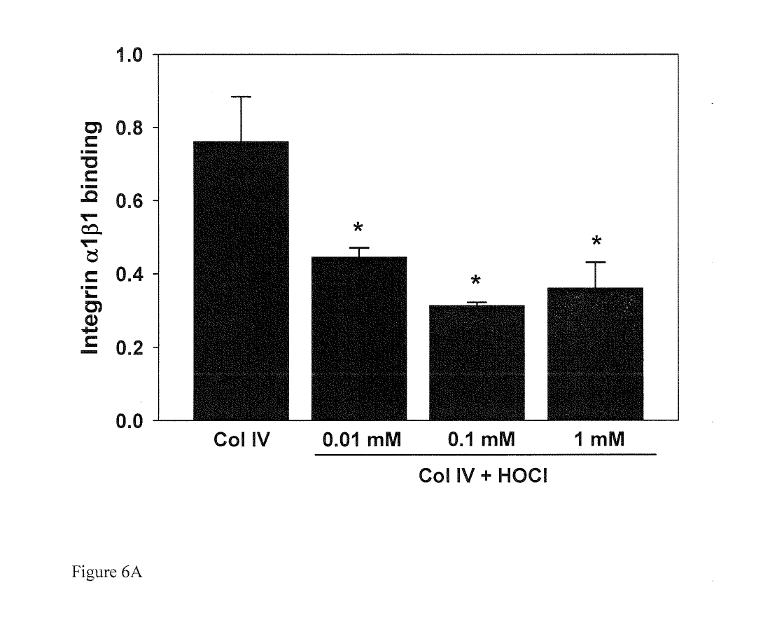

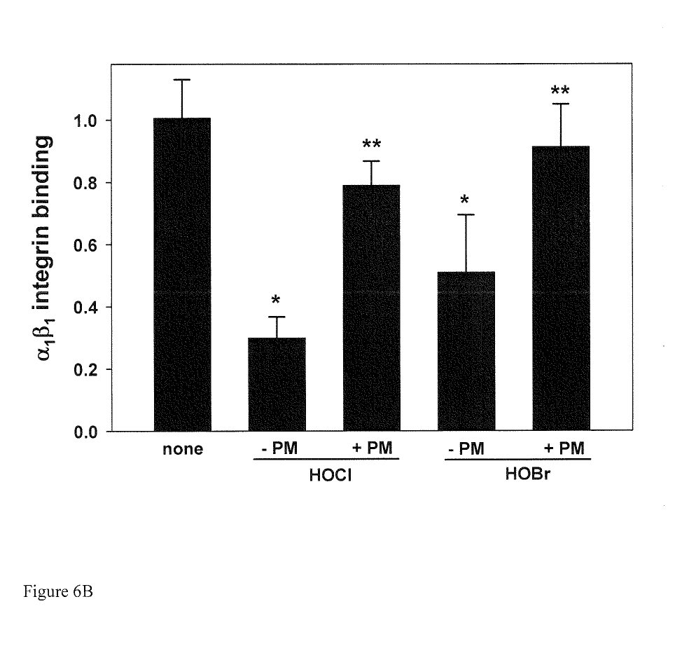

We demonstrated that hypohalous acids can inhibit RNase activity (FIG. 1). PM had higher reactivity with hypohalous acids compared to the most susceptible amino acid side chains (FIG. 2). Thus, PM inhibited halogenation of catalytically important Tyr-92 and Tyr-97 residues in RNase and protected enzymatic activity (FIG. 3A and 3B). PM reacted directly with either hypochlorous or hypobromous acid forming halogenated and oxidized PM derivatives which were identified using LC-MS/MS (FIG. 4A and 4B). PM also significantly inhibited chlorination of collagen IV by hypochlorous acid (FIG. 5) and protected binding of integrin α1β1 to collagen IV in the presence of either HOCl or HOBr (FIG. 6A and 6B). This data demonstrates that PM can protect against biomolecule damage induced by hypohalous acids, and indicates that PM can thus be used to treat a variety of inflammatory disorders in which hypohalous acids are active mediators. We further suggest that protection by PM against hypohalous acid-induced functional protein damage contributes to therapeutic effects demonstrated in PM clinical trials.

### REFERENCES

1. Zhang, C., J. Yang, and L. K. Jennings, Leukocyte-derived myeloperoxidase amplifies high-glucose-induced endothelial dysfunction through interaction with high-glucose-stimulated, vascular non-leukocyte-derived reactive oxygen species. Diabetes, 2004. 53(11): p. 2950-9.
2. Malle, E., T. Buch, and H. J. Grone, Myeloperoxidase in kidney disease. Kidney Int, 2003. 64(6): p. 1956-67.
3. Liu, B., et al., Detection of advanced oxidation protein products in patients with chronic kidney disease by a novel monoclonal antibody. Free Radic Res, 2011. 45(6): p. 662-71.
4. Xu, J., et al., Uncoupling of endothelial nitric oxidase synthase by hypochlorous acid: role of NAD(P)H oxidase-derived superoxide and peroxynitrite. Arterioscler Thromb Vasc Biol, 2006. 26(12): p. 2688-95.
5. Yang, J., et al., L-arginine chlorination results in the formation of a nonselective nitric-oxide synthase inhibitor. J Pharmacol Exp Ther, 2006. 318(3): p. 1044-9.
6. Senthilmohan, R. and A. J. Kettle, Bromination and chlorination reactions of myeloperoxidase at physiological concentrations of bromide and chloride. Arch Biochem Biophys, 2006. 445(2): p. 235-44.
7. Peterfi, Z., et al., Peroxidasin is secreted and incorporated into the extracellular matrix of myofibroblasts and fibrotic kidney. Am J Pathol, 2009. 175(2): p. 725-35.
8. Bai, Y. P., et al., Role of VPO1, a newly identified heme-containing peroxidase, in ox-LDL induced endothelial cell apoptosis. Free Radic Biol Med, 2011. 51(8): p. 1492-500.
9. Shi, R., et al., Involvement of vascular peroxidase 1 in angiotensin II-induced vascular smooth muscle cell proliferation. Cardiovasc Res, 2011. 91(1): p. 27-36.
10. Brandes, R. P., Vascular peroxidase 1/peroxidasin: a complex protein with a simple function? Cardiovasc Res, 2011. 91(1): p. 1-2.
11. Voziyan, P. A. and B.G. Hudson, Pyridoxamine as a multifunctional pharmaceutical: targeting pathogenic glycation and oxidative damage. Cell Mol Life Sci, 2005. 62(15): p. 1671-81.
12. Chetyrkin, S. V., et al., Propagation of protein glycation damage involves modification of tryptophan residues via reactive oxygen species: inhibition by pyridoxamine. Free Radic Biol Med, 2008. 44(7): p. 1276-85.
13. Chetyrkin, S., et al., Glucose Autoxidation Induces Functional Damage to Proteins via Modification of Critical Arginine Residues. Biochemistry, 2011.

### Experimental Procedures

Materials—Ribonuclease A was purchased from Worthington Biochemical Corporation. L-Lysine, Na-Acetyl-L-Lysine, NεAcetyl-L-Lysine, DL-Methionine, NAcetyl-DL-Methionine, Histidine, NaAcetyl-DL-Histidine, Imidazole, NaAcetyl-L-Arginine, NaAcetyl-L-Glutamine, NAcetyl-DL-Tryptophan, NAcetyl-DL-Tyrosine, Ribonucleic acid, Chloramine T trihydrate, Potassium Bromide, Lanthanum nitrate hydrate, Sodium Iodide, 3,3′,5,5′-Tetramethylbenzidine (TMB), N,N-Dimethyl formamide (DMF), Pyridoxamine dihydrochloride, Sodium Hypochlorite solution and type IV collagen from Engelbreth-Holm-Swarm murine sarcoma basement membrane were purchased from Sigma-Aldrich. Human Integrin α1β1 was purchased from Millipore and β1 Integrin antibody was purchased from Santa Cruz Biotechnology Inc.

Measurements of RNase Activity—RNase activity was determined by measuring the formation of acid-soluble oligonucleotide as described by Kalnitsky et al [1], with some modifications previously described by Voziyan et al [2]. For the assay, 100 μL of 3 μg/mL RNase in 100 mM sodium acetate, pH 5.0, was mixed with 100 μL of 1% yeast RNA in the same buffer. After the incubation at 37° C. for 5 min, the reaction was stopped by the addition of 100 μL of an ice-cold solution of 0.8% lanthanum nitrate in 18% perchloric acid. The incubations were kept on ice for 5 min to ensure complete precipitation of undigested RNA and then centrifuged at 12000 g for 10 min. An aliquot of the supernatant (20 μL) was diluted to 1 mL with distilled water, the amount of digested (solubilized) RNA was determined by measuring the absorbance at 260 nm. The activity of RNase incubated either alone or with PM or an amino acid substitute at 37° C. was monitored separately and used as a reference for each incubation time. This reference activity did not change significantly over the course of incubation.

Analysis of PM reactions with hypohalous acids by UPLC and ESI-MS—UPLC analyses were performed using Waters Acquity UPLC system equipped with Waters fluorescence and photodiode array detectors. The sample aliquot (10 μL) was loaded onto Phenomenex Synergy 4u Hydro-RP 80A (150×4.6 mm) column and eluted at a flow rate of 1 ml/min with the following gradient: 0-5 min, 100% buffer A; 5-15 min, linear gradient to 25% buffer B; 15-15.1 min, change to 100% buffer B; 15.1-20 min, 100% buffer B; 20-20.1 min, change to 100% buffer A; 20.1-25 min, 100% buffer A. Buffer A was 0.5% formic acid in water, buffer B was 0.5% formic acid in ACN. The UV detector was set at 295 nm. Electrospray ionization mass spectrometry (ESI-MS/MS) was performed by infusion of column eluate into Thermo TSQ mass spectrometer. Tandem MS spectra were collected at CID of 25V or 60V.

N-Chloramine Assay/ TMB Assay—The presence of N-chloramines were determined by the method of Witko et al. [3] and Dypbukt et al. [4]. The former method is based on the colorimetric measurement of triiodide ions formed by the oxidation of potassium iodide (KI) in solution. Chloroamine-T, a commercially available source of N-chloramine, was used to calibrate the assay. 100 mM chloramine-T solution was made fresh in distilled H₂O. The 100 mM chloramines-T was diluted to final concentrations ranging from 20 to 100 μM immediately before use. The direct oxidation of Kl by NaOCl was also determined and these values were subtracted as background from the corresponding treated samples. The resulting difference represented the amount of N-chloramine present in each sample. In the latter method the developing reagent was composed of 2 mM TMB in 400 mM acetate buffer, pH 5.4, containing 10% dimethlyforamide and 100 μM sodium iodide. This solution was prepared by dissolving TMB in 100% DMF, diluting with acetate buffer to get the desired final concentration of TMB, and then adding sodium iodide. When the developing reagent was mixed with the solutions of chloramines, the final concentration of TMB was always at least 10 times that of the concentration of chloramines. This prevented further oxidation of the blue product.

Cell Culture—PFHR9 cells were grown in 6 well culture treated plates and maintained at confluency for 7 days in the presence of 50 μM ascorbic acid. To remove cells from matrix, wells were briefly rinsed in 2 mL of hypotonic buffer (10 mM tri [pH 7.4], 0.1% CaCl₂, 0.1% BSA) and subsequently incubated for 10 min in 1 mL of fresh hypotonic buffer. After this, wells underwent two 5 minute washes in 1 mL hypotonic buffer +0.5% trition X-100, and then two quick washes in 1 mL hypotonic buffer +0.1% sodium deoxycholate. Sample material was returned to saline conditions by three brief washes of 2 mL each in 1X PBS. Following this procedure, wells contain plate-bound matrix that is devoid of intact cells. Cell-free plates were stored at 20° C. without PBS supernatant until use.

Chlorination of Type IV Collagen and PFHR9 Matrix Proteins—Type IV collagen from Engelbreth-Holm-Swarm (EHS) murine sarcoma basement membrane were immobilized on 96-well plates in 100 mM sodium phosphate buffer at 4° C. overnight. Plates coated with EHS collagen or with extracellular matrix deposited by PFHR9 cells were washed twice with 100 mM sodium phosphate, pH 7.5, and incubated in the same buffer with or without different concentrations of HOCl alone or HOCl and PM. Incubations were carried out for 1 hr in the dark at RT, washed twice with 100 mM sodium phosphate buffer and developed with N-Chloramine Assay for 2 h followed by detection at 340 nm.

Modification of Collagen IV and Integrin Binding Assay—20 μg/mL of type IV collagen in 20 mM sodium phosphate buffer, 7.5, was immobilized on 96 well plates at 4° C. overnight. Nonspecific binding sites were blocked with 1% BSA in TBS for 2 hr at 30° C. as previously described [5]. Wells were washed five times with either TBS or 100 mM sodium phosphate, pH 7.5, before being incubated in the same buffer with or without HOCl/HOBr alone or HOCl/l HOBr plus PM. Incubations were carried out for 1 hr in the dark at RT and later washed five times with TBS before purified α1β1 integrin was overlaid in binding buffer (TBS, 0.1% BSA, 1 mM MgCl₂, 0.2 mM MnCl₂, 5 mM octylglucoside) and incubated for 2 hr at 30° C. In some incubations, EDTA (10 mM) was added to the binding buffer. The plates were washed five times with washing buffer (TBS, 1 mM MgCl₂, 0.2 mM MnCl₂, 0.01% Tween 20) and incubated with 131 integrin antibody (4B7R,1:500) for 1 hr. After extensive washing, the bound antibodies were detected using alkaline phosphatase-conjugated anti-mouse IgG antibodies. p-Nitrophenyl phosphate substrate (Sigma) was added to the wells, and absorbance was read at 410 nm. Readings from the samples containing EDTA were subtracted from all the data to obtain the baseline corrected magnesium dependent binding.

LC-MS/MS analysis of RNase modifications—The site-specific modifications in RNase were analyzed by tandem mass spectrometry. The samples were prepared using the spin filter protocol for proteomic analysis [6]. RNase samples were digested either with trypsin. The data-dependent scanning was performed using a LTQ Orbitrap^{TM} (Thermo Fischer Scientific, San Jose, Calif.) mass spectrometer equipped with an Eksigent AS1 autosampler and an Eksigent 1D+HPLC pump attached directly to the instrument's nanospray source. The peptides were separated on a capillary tip, 100 μm×18 cm, packed with C₁₈ resin (Jupiter C₁₈, 3 μm, 300 Å, Phenomenex, Torrance, Calif.) using an inline vented trapping column that was 100 μm×6 cm. The flow rate during the solid phase extraction phase of the gradient was 2.5 μL/min, and during the separation phase it was 500 nL/min. Mobile phase A was 0.1% formic acid, while mobile phase B was acetonitrile with 0.1% formic acid. A 95 minute gradient was performed with a 15 minute washing period (100% A for the first 10 minutes followed by a gradient to 98% A at 15 minutes) to allow for removal of any residual salts. After the initial washing period, a 60 minute gradient was performed in which the first 35 minutes was a slow, linear gradient from 98% A to 75% A, followed by a faster gradient to 10% A at 65 minutes and an isocratic phase at 10% A at 75 minutes. MS/MS spectra of the peptides were obtained using data-dependent scanning in preview mode, which consisted of one full MS spectrum (mass range of 400-2000 amu) followed by five MS/MS spectra.

The data generated from the data-dependent LC-MS/MS experiments were analyzed using Sequest [7]. Peptides were matched based on the theoretical digestion of the known protein sequences. Searches were also performed for specific modifications, such as chlorinated Lys, His, and Tyr as well as oxidized His and Met; the peptide identities were confirmed by manual analysis of MS/MS spectra. The peak containing the peptide of interest was extracted from the chromatogram and the area of the peak was determined using Xcalibur™ software. The area of the peak containing modified peptide was normalized to a reference peptide angiotensin I added to the sample prior to the experiment.

### REFERENCES

1. Kalnitsky, G. and H. Resnick, The effect of an altered secondary structure on ribonuclease activity. J Biol Chem, 1959. 234(7): p. 1714-7.
2. Voziyan, P. A., et al., A post-Amadori inhibitor pyridoxamine also inhibits chemical modification of proteins by scavenging carbonyl intermediates of carbohydrate and lipid degradation. J Biol Chem, 2002. 277(5): p. 3397-403.
3. Witko, V., A. T. Nguyen, and B. Descamps-Latscha, Microtiter plate assay for phagocyte-derived taurine-chloramines. J Clin Lab Anal, 1992. 6(1): p. 47-53.
4. Dypbukt, J. M., et al., A sensitive and selective assay for chloramine production by myeloperoxidase. Free Radic Biol Med, 2005. 39(11): p. 1468-77.
5. Pedchenko, V., R. Zent, and B. G. Hudson, Alpha(v)beta3 and alpha(v)beta5 integrins bind both the proximal RGD site and non-RGD motifs within noncollagenous (NCI) domain of the alpha3 chain of type IV collagen: implication for the mechanism of endothelia cell adhesion. J Biol Chem, 2004. 279(4): p. 2772-80.
6. Manza, L. L., et al., Sample preparation and digestion for proteomic analyses using spin filters. Proteomics, 2005. 5(7): p. 1742-5.
7. Liebler, D. C., Proteomic approaches to characterize protein modifications: new tools to study the effects of environmental exposures. Environ Health Perspect, 2002. 110 Suppl 1: p. 3-9.

We claim:

1. A method for treating an inflammatory disorder, comprising administering to a subject suffering from an inflammatory disorder thereof an amount of pyridoxamine, or a pharmaceutically acceptable salt thereof, to treat the inflammatory disorder.

2. A method for limiting development of an inflammatory disorder, comprising administering to a subject at risk of developing an inflammatory disorder an amount of pyridoxamine, or a pharmaceutically acceptable salt thereof, to limit development of the inflammatory disorder.

3. The method of claim 1, wherein the subject has, or is at risk of developing, an inflammatory disorder selected from the group consisting of cystic fibrosis, asthma, rheumatoid arthritis, multiple sclerosis, Parkinson's disease, inflammatory bowel disease, irritable bowel syndrome, ulcerative colitis, vasculitis, and Crohn's disease.

4. The method of claim 1, wherein between 50 mg/day and 2000 mg/day of the pyridoxamine, or pharmaceutically acceptable salt thereof, is administered to the subject.

5. The method of claim 1, wherein the method further comprises administering a second anti-inflammatory compound to the subject.

6. The method of claim 1 wherein the pyridoxamine, or pharmaceutically acceptable salt thereof, is administered orally.

7. The method of claim 1, wherein the subject has been identified as producing excess hypohalous acid compared to a control.

8. The method of claim 7, wherein the hypohalous acid is selected from the group consisting of hypochlorous acid (HOCl) and hypobromous acid (HOBr).

9. The method of claim 2, wherein the subject has, or is at risk of developing, an inflammatory disorder selected from the group consisting of cystic fibrosis, asthma, rheumatoid arthritis, multiple sclerosis, Parkinson's disease, inflammatory bowel disease, irritable bowel syndrome, ulcerative colitis, vasculitis, and Crohn's disease.

10. The method of claim 2, wherein between 50 mg/day and 2000 mg/day of the pyridoxamine, or pharmaceutically acceptable salt thereof, is administered to the subject.

11. The method of claim 2, wherein the method further comprises administering a second anti-inflammatory compound to the subject.

12. The method of claim 2 wherein the pyridoxamine, or pharmaceutically acceptable salt thereof, is administered orally.

13. The method of claim 2, wherein the subject has been identified as producing excess hypohalous acid compared to a control.

14. The method of claim 13, wherein the hypohalous acid is selected from the group consisting of hypochlorous acid (HOCl) and hypobromous acid (HOBr).

15. A method for treating or limiting development of an inflammatory disorder, comprising:
(a) identifying a subject with an inflammatory disorder that produces excess hypohalous acid compared to control; and
(b) treating the subject that produces excess hypohalous acid with an amount of pyridoxamine, or a pharmaceutically acceptable salt thereof, to treat the treat or limit development of the inflammatory disorder.

16. The method of claim 15, wherein the hypohalous acid is selected from the group consisting of HOCl and HOBr.

17. The method of claim 7, wherein the amount of hypohalous acid produced by the subject is measured in a urine sample obtained from the subject.

18. The method of claim 13, wherein the amount of hypohalous acid produced by the subject is measured in a urine sample obtained from the subject.

19. The method of claim 15, wherein the amount of hypohalous acid produced by the subject is measured in a urine sample obtained from the subject.

20. A method for monitoring pyridoxamine therapy in a subject, comprising
(a) analyzing a urine sample obtained from a subject with an inflammatory disorder being treated with pyridoxamine, or a pharmaceutically acceptable salt thereof, for the presence of pyridoxamine adducts with hypohalous acids; and
(b) comparing an amount of pyridoxamine-hypohalous adducts in the sample against a standard; and
(c) determining efficacy of the pyridoxamine therapy to treat the inflammatory disorder based on the comparison.
